(19) United States
(12) Patent Application Publication
(10) Pub. No.: US 2022/0305178 A1
(43) Pub. Date: Sep. 29, 2022

(54) CANCER TREATMENT BY IN VITRO AMINO ACID DEPRIVATION

(71) Applicant: YUANHENG BIOMEDICAL CO., LTD., New Taipei City (TW)

(72) Inventors: Tsung-Ping Shih, New Taipei City (TW); Shuo-Ming Hsu, New Taipei City (TW); Hsin-Chien Huang, New Taipei City (TW); Kuan-Yu Liu, New Taipei City (TW)

(21) Appl. No.: 17/210,926

(22) Filed: Mar. 24, 2021

(51) Int. Cl. A61M 1/16; G01N 33/50

(52) U.S. Cl. CPC A61M 1/1654; G01N 33/5044; A61M 2202/0413; A61M 2202/0415; A61M 2202/04

(57) ABSTRACT

Provided is a method for cancer treatment in a patient comprising extracorporeal dialysis of blood, plasma or peritoneal fluid of the patient with a dialysis system for removing a target amino acid, and the dialysis system comprising: a dialysis machine, a dialyzer having a dialysis membrane, and a dialysate; wherein the dialyzer is connected to the dialysis machine and the dialysate flows within the dialysis machine and the dialyzer; and an enzyme for degrading the target amino acid is provided to the dialysis membrane and/or the dialysate, and the target amino acid includes asparagine, glutamine, arginine, serine, methionine or any combination thereof. By modifying dialysis to achieve in vitro amino acid deprivation and incorporating personalized diagnosis, the present invention not only provides a novel precision medicine with better anticancer efficacy and less side effects, but also requires less time and cost for development.

### BACKGROUND OF THE INVENTION

### 1. Field of the Invention

The present invention relates to a method for cancer treatment, especially to a method for cancer treatment by personalized diagnosis and precision medicine.

### 2. Description of the Prior Arts

Cancer is the second most common cause of death in the United States. 1.9 million new cancer cases are estimated to be diagnosed in 2020 and the death number for cancer is predicted to be 0.6 million. Hence, cancer poses great threat to the public health and heavy financial burden to patients. While traditional cancer therapies including surgery, chemotherapy, radiotherapy and immunotherapy are commonly adopted, new approaches for cancer treatment are under continuous development, for example, the important role of amino acids in cancer metabolism is dissected and utilized for new drug discovery.

Three drugs targeting the amino acid for cancer treatment are approved by the US Food and Drug Administration (FDA) for legal uses. Asparaginase with the trade name of Elspar is the first-generation drug approved in 1978, and the distribution of Elspar was halted in December 2012. Oncaspar is the second-generation drug made by ENZON Pharmaceuticals in 2006, and the active ingredient is a covalent conjugate of Escherichia coli-derived L-asparaginase with monomethoxypolyethylene glycol. The indication of Oncaspar is as a component of antineoplastic combination therapy in acute lymphoblastic leukaemia (ALL) in pediatric patients from birth to 18 years, and adult patients. ERWINAZE is the third-generation drug approved in 2011, and the active ingredient is asparaginase Erwinia chrysanthemi. The indication of ERWINAZE is as a component of a multi-agent chemotherapeutic regimen for the treatment of patients with acute lymphoblastic leukemia (ALL) who have developed hypersensitivity to E. coli-derived asparaginase.

New drug discovery is expensive and requires years for clinical trials. Also, the drug circulating in the patient's body could cause undesirable side effects, for example, hypersensitivity. Hence, a new approach for cancer treatment which is safer and requires less cost and time for development is to be proposed.

### SUMMARY OF THE INVENTION

In consideration of the drawbacks of the cancer treatment currently available, one objective of the present invention is to provide a cancer treatment which is safer and requires less cost and time for development.

Another objective of the present invention is to modify dialysis and integrate the dialysis with personalized diagnosis to propose a novel precision medicine with better anticancer efficacy and less side effects.

The present invention provides a new approach to deplete one or more amino acids in the patient's blood, plasma or peritoneal fluid to inhibit the growth and/or regeneration of cancer cells.

In order to achieve the above objectives, the present invention provides a method for cancer treatment in a patient comprising extracorporeal dialysis of blood, plasma or peritoneal fluid of the patient with a dialysis system for removing a target amino acid, and the dialysis system comprising: a dialysis machine, a dialyzer having a dialysis membrane, and a dialysate; wherein the dialyzer is connected to the dialysis machine and the dialysate flows within the dialysis machine and the dialyzer; and an enzyme for degrading the target amino acid is provided to the dialysis membrane and/or the dialysate, and the target amino acid includes, but is not limited to, asparagine, glutamine, arginine, serine, methionine or any combination thereof.

In the present invention, an enzyme for degrading the target amino acid refers to at least one enzyme. If there are multiple target amino acids, each of the specific corresponding enzymes is adopted.

The extracorporeal dialysis of blood, plasma or peritoneal fluid of the patient means the dialysis is carried out in the dialysis machine outside the blood circulation system of the patient, and the dialysis machine may be imbedded in the body of the patient or set outside the body of the patient.

In one embodiment, the dialysis system further comprises a supplement fluid having the enzyme for degrading the target amino acid. Said supplement fluid provides the enzyme for degrading the target amino acid to the dialysate.

In another embodiment, the dialysis membrane has two surfaces opposite to each other, and the enzyme for degrading the target amino acid is immobilized or coated on the surface that contacts the dialysate.

During extracorporeal dialysis of blood, plasma or peritoneal fluid, the molecule with the molecular weight less than 1000 Da (1 kDa) could pass through the dialysis membrane and the enzyme, for example, asparaginase with a molecular weight about 35 kDa cannot pass through the dialysis membrane. As amino acids have an average molecular weight of 110 Da, for example, asparagine has a molecular weight of 132 Da, asparagine could leave the blood and be subjected to the degradation by asparaginase in the dialysates or on the surface that contacts the dialysates during dialysis, while asparaginase is refrained from entering the blood, plasma or peritoneal fluid of patients. As the exogenous enzyme for amino acid degradation, e.g. asparaginase or a fusion protein will not enter the circulation system of the patient, the undesirable side effect or hypersensitivity induced by exogenous protein will not happen in the present invention.

Preferably, the dialysate is in an amount of 1 time to 9 times of the amount of the blood, the plasma or the peritoneal fluid of the patient, for example, 1 time, 2 times, 3 times, 4 times, 5 times, 6 times, 7 times, 8 times or 9 times. More preferably, the dialysate is in an amount of 2 times to 4 times of the amount of the blood, the plasma or the peritoneal fluid of the patient.

Preferably, the flow rate or circulation speed of the dialysate was 200 ml/min to 600 ml/min, for example, 200 ml/min, 250 ml/min, 300 ml/min, 350 ml/min, 400 ml/min, 450 ml/min, 500 ml/min, 550 ml/min or 600 ml/min.

Preferably, the concentration of the enzyme for degrading the target amino acid in the dialysate is more than 0.1 K.U./ml. More preferably, the concentration of the enzyme for degrading the target amino acid in the dialysate is 0.1 K.U./ml to 2 K.U./ml, for example, 0.1 K.U./ml, 0.5 K.U./ml, 0.8 K.U./ml, 1.0 K.U./ml, 1.2 K.U./ml, 1.5 K.U./ml or 2 K.U./ml.

Preferably, the dialyzer is, but not limited to, a low flux dialyzer or a high flux dialyzer. More preferably, the dialyzer is a high flux dialyzer.

Preferably, the dialyzer has an effective membrane area more than 0.5 m². More preferably, the effective membrane area is 0.5 m² to 2.4 m², for example, 0.5 m², 0.8 m², 1.0 m², 1.2 m², 1.4 m², 1.6 m², 1.8 m², 2.0 m², 2.2 m² or 2.4 m².

Preferably, the dialysis membrane is made of one or more materials selected from, but not limited to, the group consisting of cellulose triacetate, ethylenevinylalvohol copolymer, polyarylethersulfone, polyarylethersulfone-polyamide, polyester-polymer alloy, polyethersulfone, polymethylmethacrylate, polysulfone and polypropylene.

Preferably, the target amino acid is selected from a group consisting of asparagine, glutamine, arginine, serine and methionine.

The removal of the target amino acid could be carried out simultaneously or in turn by the extracorporeal dialysis of blood of the patient.

As the proliferation of cancer cells requires more protein synthesis than that of normal cells, and amino acids serve as the fundamental unit of protein, deprivation of essential amino acids, such as methionine, could inhibit the growth of cancer cells.

Preferably, the concentration of the target amino acid in the blood, plasma or peritoneal fluid of the patient decreases to below the desirable concentration that is estimated to incur cell death of cancer cells after the extracorporeal dialysis. Such concentration could be adjusted according to the profile from the amino acid deprivation test of the present invention. More preferably, after a tumor tracking process of the present invention is completed, the concentration of the target amino acid in the blood, plasma or peritoneal fluid of the patient decreases to below the concentration that is shown to incur cell death of cancer cells based on the result of the tumor tracking process.

Further, while the cancer cell may be subjected to mutation and lacks the ability to assimilate particular enzyme for amino acid metabolism, deprivation of particular amino acid could specifically induce the death of the cancer cell without undesirable influences on normal cells. For example, argininie deiminase (ADI) could turn arginine into citruline, and citruline could be turned into arginine by argininosuccinate synthase1 (ASS) and argininosuccinate lyase (ASL) in normal cells. If the cancer cell has the gene deficit of ASS, the cycle to assimilate arginine will be disrupted, which means the lower concentration of arginine will cause specific starvation of the cancer cell and lead the cancer cell to death.

Preferably, the cancer is selected from, but not limited to, a group consisting of acute lymphoblastic leukemia (ALL), acute myeloid leukemia (AML), small cell lung cancer, lung mesothelioma, chest wall tumor, abdomen-pelvis tumor, small intestinal tumor, thymoma, mediastinal tumor, prostate cancer, cervical cancer, endometrial cancer, ovarian cancer, uterine sarcoma blood cancer, lymphoma, pancreatic cancer, sarcoma, brain cancer, low-grade astrocytoma, high-grade astrocytoma, pituitary adenoma, meningioma, CNS lymphoma, oligodendroglioma, brain stem tumor, ependymoma, breast cancer, colorectal cancer, liver cancer, liver adenocarcinoma, gallbladder cancer, biliary tract cancer, head and neck cancer, nasopharyngeal cancer, esophageal cancer, craniopharyngioma, laryngeal cancer, oropharyngeal cancer, salivary gland tumor, hypopharyngeal cancer, thyroid cancer, oral cavity tumor, kidney cancer, bladder cancer, anal cancer and skin cancer.

In one embodiment, the present invention further comprises a tumor analysis before the extracorporeal dialysis, and the tumor analysis comprises the steps of:

(A1) obtaining a cancer cell from the patient;

(A2) cultivating the cancer cell to obtain a cell culture; and

(A3) conducting an amino acid deprivation test to obtain a profile indicating the type of the target amino acid along with the corresponding concentration and period for cell death of the cell culture.

The cell culture refers to the culture of the primary cells obtained from the patient. Preferably, the cell culture may be purified to obtain a cell line or multiple cell lines.

By the tumor analysis, the cancer cell from the patient will be studied, and its unique profile will be helpful for precise removal of the target amino acid to which the autologous cancer cell is particularly vulnerable. Also, the period required for cell death of the cell culture and the lethal low level of amino acids concentration in the cell culture will also be taken as a critical reference to set the parameters for extracorporeal dialysis of blood, for example, the clearance rate of amino acid in the blood of the patient.

Preferably, the cancer cell is, but not limited to, a circulating tumor cell (CTC) or is from biopsy. More preferably, the cancer cell is a circulating tumor cell. The circulating tumor cells (CTCs) could be isolated without surgery, so isolation and collection of CTCs is less invasive and safer than biopsy.

Preferably, the cell culture is, but not limited to, a 2D or 3D cell culture. More preferably, the cell culture is a 3D cell culture. As the 3D cell culture provides the microenvironment more similar to the tumor in patients, the information drawn from the profile derived from the 3D cell culture will be more precise than that from the 2D cell culture.

In another embodiment, the present invention further comprises a pattern analysis of blood amino acid concentration fluctuation for the target amino acid before the extracorporeal dialysis.

Preferably, the pattern analysis of blood amino acid concentration fluctuation for the target amino acid is generally conducted once. More preferably, the patient follows the instructions the same as those provided in the nutrition management of the present invention.

The pattern of blood amino acid concentration fluctuation of the patient, especially the unique concentration fluctuation of the target amino acid in blood after meals, will be taken as a critical reference to set the parameters for extracorporeal dialysis of blood.

In one embodiment, the present invention further comprises nutrition management during the cancer treatment of the patient.

The goal of the nutrition management is to control the blood concentration fluctuation of the target amino acid of the patient after meals to stay within an acceptable range. Preferably, the nutrition management will not remove the food releasing the target amino acid from daily diet of the patients completely.

In another embodiment, the present invention further comprises a blood monitoring process for the concentration fluctuation of the target amino acid during the cancer treatment of the patient.

Preferably, the blood monitoring process is carried out in a real time manner during the extracorporeal dialysis and/or periodically after or before the extracorporeal dialysis during the treatment of cancer.

In the alternative, the pattern analysis of blood amino acid concentration fluctuation for the target amino acid as mentioned above will indicate the timing of the target amino acid to be released into the blood of the patient after a meal or the approximate digestion time, so the blood monitoring process may be carried out at the time based on the result of the pattern analysis of blood amino acid concentration fluctuation for the target amino acid.

Preferably, the blood monitoring process is carried out 60 minutes after a meal and/or whenever the extracorporeal dialysis is completed. More preferably, the result of the target amino acid concentration is obtained 1 hour to 2 hours after a meal.

In another embodiment, the present invention further comprises a tumor tracking process during the cancer treatment of the patient.

Preferably, the tumor tracking process comprises Circulating Tumor Cell (CTC) analysis, imaging examination by X-ray, ultrasound, Magnetic Resonance Imaging (MRI) or Computed Tomography (CT).

Preferably, the extracorporeal dialysis is carried out whenever such extracorporeal dialysis is required or suggested according to the result of the pattern analysis of blood concentration fluctuation of the target amino acid, the result of the blood monitoring process, the result of the nutrition management, or the result of the tumor tracking process.

Preferably, the extracorporeal dialysis is carried out 1 hour after a meal. More preferably, the extracorporeal dialysis is carried out 1 hour to 2 hours after a meal.

Preferably, the tumor tracking process is carried out whenever the extracorporeal dialysis is completed.

Preferably, the extracorporeal dialysis requires 3 minutes to 1 hour for each time, .e.g. 5 minutes for each time, 10 minutes for each time, 15 minutes for each time, 20 minutes for each time, 25 minutes for each time, 30 minutes for each time, 40 minutes for each time, 50 minutes for each time or 60 minutes for each time. More preferably, the extracorporeal dialysis requires 30 minutes to 1 hour for each time.

The extracorporeal dialysis is carried out stage by stage. The patient will receive extracorporeal dialysis until the cell death of cancer cells is achieved, or the tumor is ameliorated, shrunk or removed.

In one embodiment, the extracorporeal dialysis lasts for a period based on the result of the tumor tracking process. Preferably, the extracorporeal dialysis is carried out periodically or irregularly and lasts for 3 days to 2 weeks.

In one embodiment, the frequency of extracorporeal dialysis is based on the situation of the patient, e.g. the concentration fluctuation of the target amino acid in the blood of the patient, or the result of the tumor tracking process. Preferably, the frequency of extracorporeal dialysis may be three times for each day to 3 days or 4 days for one time.

According to the present invention, the following disadvantages of the current medicine could be avoided or reduced: (1) allergic reaction induced by exogenous enzymes; (2) decomposition and removal of the enzyme for amino acid degradation in a gradual manner during blood circulation of the patient; (3) concentration fluctuation of amino acids in blood; (4) failure to adjust the effective amount of enzyme for amino acid degradation precisely; (5) drug resistance; (6) few or limited types of cancer to be targeted for the indication.

In one embodiment, the present invention provides a method for personalized cancer treatment by removing a target amino acid in a patient through integrating a method for personalized cancer diagnosis and in vitro amino acid deprivation. The method for personalized cancer treatment by removing a target amino acid in a patient comprises:

a tumor analysis comprising the steps of:

(A1) obtaining a cancer cell from the patient;

(A2) cultivating the cancer cell to obtain a cell culture; and

(A3) conducting an amino acid deprivation test to obtain a profile indicating the type of the target amino acid along with the corresponding concentration and period for cell death of the cell culture, wherein the target amino acid includes, but is not limited to, asparagine, glutamine, arginine, serine, methionine or any combination thereof;

a pattern analysis of blood concentration fluctuation of the target amino acid;

extracorporeal dialysis of blood, plasma or peritoneal fluid of the patient with a dialysis system for removing the target amino acid, and the dialysis system comprising: a dialysis machine, a dialyzer having a dialysis membrane, and a dialysate; wherein the dialyzer is connected to the dialysis machine and the dialysate flows within the dialysis machine and the dialyzer; and an enzyme for degrading the target amino acid is provided to the dialysis membrane and/or the dialysate;

a blood monitoring process for the concentration of the target amino acid;

nutrition management during the cancer treatment of the patient; wherein the pattern analysis is carried out before the extracorporeal dialysis, and the extracorporeal dialysis is carried out stage by stage; and a tumor tracking process during the personalized cancer treatment of the patient.

Preferably, the method for personalized cancer treatment by removing a target amino acid in a patient comprises the method for cancer treatment in a patient as mentioned above.

In another embodiment, the present invention provides a dialysis system comprising: a dialysis machine, a dialyzer having a dialysis membrane, and a dialysate; wherein the dialyzer is connected to the dialysis machine and the dialysate flows within the dialysis machine and the dialyzer; and an enzyme for degrading a target amino acid is provided to the dialysis membrane and/or the dialysate, and the target amino acid includes, but is not limited to, asparagine, glutamine, arginine, serine, methionine or any combination thereof.

In one embodiment, the present invention provides a dialysis membrane for dialysis having an enzyme for degrading a target amino acid, wherein the target amino acid includes, but is not limited to, asparagine, glutamine, arginine, serine, methionine or any combination thereof.

Preferably, the dialysis comprises hemodialysis, peritoneal dialysis or plasma dialysis.

In another embodiment, the present invention provides a dialysate for dialysis having an enzyme for degrading a target amino acid, wherein the target amino acid includes, but is not limited to, asparagine, glutamine, arginine, serine, methionine or any combination thereof.

Preferably, the dialysis comprises hemodialysis, peritoneal dialysis or plasma dialysis.

Finally, dialysis is widely applied for renal failure, so the present invention not only requires less period and cost for development, but also provides safer and better treatment for the patient and requires shorter treatment duration in comparison with the conventional cancer therapy. Hence, the present invention is beneficial and affordable for patients and reduces the burden of public health for cancer treatment.

### BRIEF DESCRIPTION OF THE DRAWINGS

FIG. 1 is a schematic diagram showing the extracorporeal dialysis system of the present invention.

FIG. 2 is a schematic diagram showing the fluctuation of blood amino acid concentration of the patient.

### DETAILED DESCRIPTION OF THE PREFERRED EMBODIMENTS

In the following, several examples are showed to demonstrate the present invention. One person skilled in the art can easily understand the merits and effects through these examples. It should be understood that the examples in the specification are only for the purpose of illustrating the implementation of the present invention, but shall not be used to limit the scope of the present invention. One person skilled in the art can make necessary changes or modifications to implement or apply the content of the present invention without departing from the spirit of the present invention.

### EXAMPLE 1

### Modified Hemodialysis

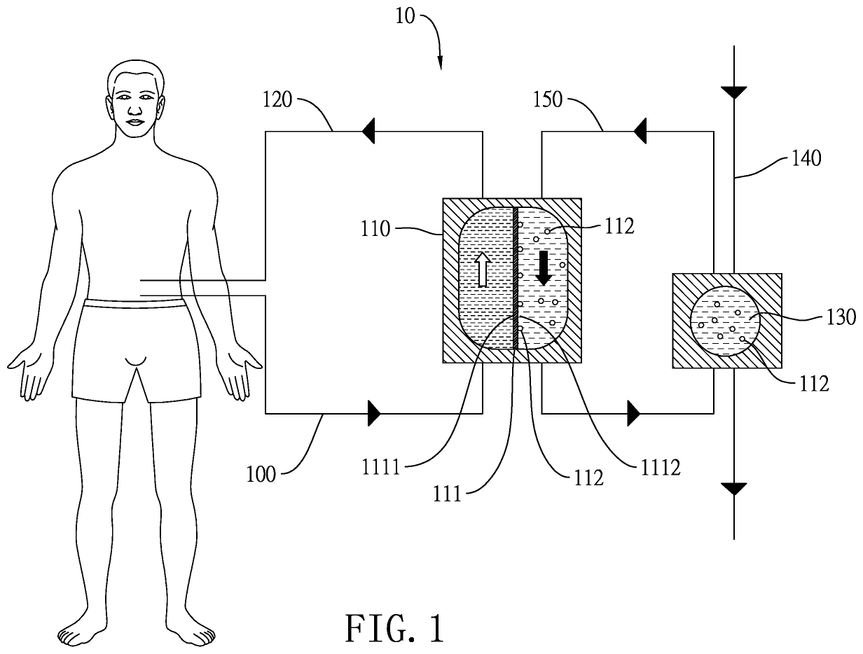

FIG. 1 shows an extracorporeal dialysis system 10 of one embodiment of the present invention in a simple configuration whereby the blood of a patient is taken out of the body through a blood line 100, pumped by the pump (not shown) through a dialyzer 110 and back into the patient through another blood line 120. Dialysates 130 are pumped to flow in the direction opposite to that of the blood in the dialyzer 110. The dialyzer 110 has a dialysis membrane 111, and the dialysis membrane 111 has two surfaces 1111 and 1112 opposite to each other. The surface 1111 contacts the blood and the surface 1112 contacts the dialysates 130. Further, enzymes 112 for amino acid degradation are immobilized or coated on the surface 1112 that contacts the dialysates 130 to refrain the enzymes 112 for amino acid degradation from entering the blood of the patient.

In the alternative, the enzymes 112 for amino acid degradation could be added in the dialysates 130 in advance or added by a supplement line 140 during the operation of the dialysis system 10.

All amino acids in the patient's blood could pass the dialysis membrane 111 and only the target amino acid will be decomposed by the enzymes 112 for amino acid degradation. As mass transport across the dialysis membrane 111 will tend to equilibrate concentrations, the concentration of any molecular species in the patient's blood will approach the same concentration in the dialysates 130 of the same species. Hence, while the concentration of the target amino acid in the patient's blood is higher than that in the dialysates 130 in the dialyzer 110, the target amino acid in the patient's blood will keep moving to the dialysates 130 and be removed from the patient's blood so as to reduce the concentration of the target amino acid in the patient's blood.

The dialysates 130 are provided to the dialyzer 110 by the circulation pipeline 150 of a dialysis machine (not shown). The dialysates 130 could be further filtered to remove the wastes derived from the degradation of amino acid and from the patient's blood. In the alternative, the dialysates 130 are non-recyclable and discarded after leaving the dialyzer 110.

The dialysis membrane 111 is a flat membrane type. Preferably, the dialyzer 110 is a high flux dialyzer and the dialysis membrane 111 is a hollow fiber type with the patient's blood moving through the fibers, and the extrafibrilar space between the hollow fibers in the dialyzer 110 is filled with and flushed by the dialysates 130. The flow of the dialysates 130 in the extrafibrilar space is in the opposite direction from that of the patient's blood in the fiber. The wall of the fiber allows passage of substances with low molecular weight only, e.g. water and amino acids.

In the present embodiment, the dialysates 130 are a normal saline. Preferably, the dialysates 130 are further supplemented with other ingredients or nutrients. More preferably, the dialysates 130 are added with amino acids except the target amino acid.

### EXAMPLE 2

### Modified Hemodialysis

Example 2 is carried out as follows:

1. Simulated blood:

900 ml of pig's blood and 100 ml of Gibco Roswell Park Memorial Institute (RPMI) 1640 Medium (RPMI 1640 Medium) were mixed to obtain a diluted pig's blood. The diluted pig's blood was further added with 1.8 mg/ml EDTA-K²⁺, 0.05% sodium azide; and 132 μl of Heparin with the concentration of 5000 U/ml to obtain a simulated blood, wherein the concentration of EDTA-K²⁺ of the simulated blood was 16 mg/ml.

2. Setting for hemodialysis:

The dialysis machine is Toray TR-321. The dialysate for extracorporeal circulation was triple the amount of the simulated blood and was 3000 ml, and the flow rate of dialysate was 500 ml/min. Before the simulated blood entered the dialysis machine, 1X PBS was used to clean the circulation pipeline of the dialysis machine, to achieve the required balance of the artificial kidney (Polyflux 17L), and to get rid of all bubbles in the artificial kidney, and the circulation speed was 250 ml/min. Besides, the effective membrane area of the artificial kidney is 1.7 m².

The dialysate consisted of Buffer A (hemodialysis concentrate No. 16, purchased from Taiwan Biotech Co., LTD), Buffer B (Sodium Bicarbonate Concentrate 8.4%, purchased from Fressenius Medical Care Ag, 61348 Bad Homburg, Germany) and R.O. water, wherein the volume ratio of Buffer A, Buffer B and R.O. water is 1:1.225:32.775.

The target amino acid is Asparagine (Asn), and the rest are non-target amino acids. The 3000 ml dialysate was added with 3 ml of Asparaginase with the concentration of 1000 K.U./ml (LEUNASE®) in the Experimental group 1, so the concentration of Asparaginase in the dialysate is 1 K.U./ml. No Asparaginase was added in the Control group 1.

The conductivity of the dialysate was adjusted to about 14 mS/cm. The dialysis temperature was set to be 37° C. When the pressure of the circulation pipeline, the temperature and the conductivity of the dialysate reached the desirable value and were stable, a green light signaled to indicate that hemodialysis was ready. The simulated blood was pumped into the circulation pipeline and entered the artificial kidney. Besides, the simulated blood was further added with Heparin with the concentration of 5000 U/ml at the flow rate of 4.4 ml/hour before the simulated blood entered the artificial kidney. The concentrations of amino acids in the simulated blood before and after dialysis were detected by ultra performance liquid chromatography (UPLC) and the results were shown in Table 1 and Table 2.

TABLE 1
The concentrations (μM) of amino acids of the
simulated blood of the Control group 1

| | Retention | Dialysis time (min) | | | |
|---|---|---|---|---|---|
| types | Time | 0 | 10 | 20 | 30 |
| Asn | 2.14 | 22.3 | 9.9 | N/A | N/A |
| Ser | 2.854 | 147.8 | 43.1 | 27.2 | 25.3 |
| Gln | 3.006 | 204.5 | 49.5 | 30.9 | 22.2 |
| Arg | 3.119 | 111.0 | 27.2 | 16.4 | 13.8 |
| Met | 7.373 | 33.3 | 9.7 | 5.8 | 5.0 |

According to Table 1, the concentrations of all amino acids were lowered due to the equilibrium between the dialysate and the simulated blood. Further, Asparagine (Asn) is undetectable within 20 minutes of dialysis, and the limit of detection (LOD) of Asparagine was 5 μM.

TABLE 2
The concentrations (μM) of amino acids of
the simulated blood of the Experimental group 1

| | Retention | Dialysis time (min) | | | | | |
|---|---|---|---|---|---|---|---|
| types | Time | 0 | 3 | 5 | 10 | 20 | 30 |
| Asn | 2.1 | 41.8 | 32.3 | 24.2 | N/A | N/A | N/A |
| Ser | 2.9 | 159.8 | 67.7 | 63.2 | 17.5 | 20.0 | 17.8 |
| Gln | 3.0 | 381.8 | 147.5 | 131.5 | 24.2 | 18.2 | 18.3 |
| Arg | 3.1 | 212.4 | 83.0 | 74.6 | 16.9 | 14.7 | 14.3 |
| Met | 7.4 | 38.5 | 16.0 | 14.4 | 3.7 | 3.8 | 3.5 |

According to Table 2, due to the addition of Asparaginase, Asparagine (Asn) is undetectable within 10 minutes of dialysis, even the original concentration of Asparagine in the Experimental group 1 is higher than that in the Control group 1. Hence, by adding an enzyme for degrading the target amino acid, the removal of the target amino acid could be speeded up. Further, as the extracorporeal dialysis could be completed earlier after adding the enzyme for degrading the target amino acid, the reduction of non-target amino acids in the blood by equilibrium will be alleviated so as to lower the risk of incurring side effect derived from the removal of non-target amino acids.

### EXAMPLE 3

### Modified Peritoneal Dialysis

Example 3 is carried out as follows:

1. Preparation of peritoneal fluid:

RPMI 1640 Medium was ten times diluted to obtain 1 L of RPMI 1640 diluent with Dianeal PD-2 (Peritoneal Dialysis Solution) and to serve as a peritoneal fluid for the control group 2. 1 L and 2 L of the same RPMI 1640 diluent was prepared for the Experimental group 2 and Experimental group 3, respectively.

2. Setting for peritoneal dialysis:

The dialysis machine (Toray TR-321) had a circulation volume for a dialysate which was the same amount of the RPMI 1640 diluent, and the volume ratio for the peritoneal fluid and dialysate is 1:1 for the control group 2 and the experimental groups 2 & 3. The flow rate or circulation speed of the dialysate was 250 ml/min. Before the peritoneal fluid (RPMI 1640 diluent) entered the dialysis machine, RPMI 1640 diluent was also used to clean the circulation pipeline of the dialysis machine, to achieve the required equilibrium of the artificial kidney, and to get rid of all bubbles in the artificial kidney. Besides, the artificial kidneys of the control group 2 and the experimental group 2 were NV-15U with the effective membrane area of 1.5 m²; and the artificial kidney of the experimental group 3 was NV-21U with the effective membrane area of 2.1 m².

The dialysate is DIANEAL PD-2 Peritoneal Dialysis Solution with 1.5% Dextrose (Baxter). The target amino acid is Asparagine (Asn), and the concentration of Asparaginase in the dialysate is 1 K.U./ml in the Experimental groups 2 & 3. No Asparaginase was added in the control group 2.

The dialysis temperature was set to be 37° C. The peritoneal fluid (RPMI 1640 diluent) was first pumped into slot A for temporary storage and then entered the artificial kidney. After peritoneal fluid (RPMI 1640 diluent) left the artificial kidney, peritoneal fluid (RPMI 1640 diluent) further entered slot B for temporary storage, and then left the dialysis machine, e.g. the peritoneal fluid (RPMI 1640 diluent) was pumped back to the peritoneal cavity of the patient. The concentrations of amino acids in the peritoneal fluid before and after dialysis were studied by using Amino Acid Analysis Systems (Waters UPLC® Amino Acid Analysis (AAA) Solution) and the result was shown in Table 3 to Table 5.

TABLE 3
The concentrations (μM) of amino acids
of the peritoneal fluid of the control group 2

| | Dialysis time (min) | | | | | | |
|---|---|---|---|---|---|---|---|
| types | 0 | 3 | 5 | 10 | 20 | 30 | 60 |
| Asn | 46.276 | 33.144 | 27.376 | 17.28 | 19.86 | 16.02 | 16.036 |
| Arg | 101.5 | 81.1 | 66.7 | 39.6 | 46.9 | 38.2 | 38.4 |

Arginine (Arg) serves as a reference to know the decreasing rate of a non-target amino acid.

According to Table 3, the concentration of both amino acids were lowered due to the equilibrium between the dialysate and the peritoneal fluid.

TABLE 4
The concentrations (μM) of amino acids of the
peritoneal fluid of the experimental group 2

| | Dialysis time (min) | | |
|---|---|---|---|
| types | 0 | 3 | 5 |
| Asn | 27.284 | 23.254 | 9.088 |
| Arg | 102.6 | 90.0 | 70.5 |

According to Table 4, due to the addition of Asparaginase, the concentration of Asparagine (Asn) is reduced to be less than 50% within 5 mins. In contrast, according to Table 3, the concentration of Asn is still more than 50% within 5 mins. Hence, by adding an enzyme for degrading the target amino acid, the removal of the target amino acid could be speeded up. Further, the decreasing rates of Arg in Table 3 and Table 4 are similar, which means the side effect derived from the removal of non-target amino acids could be lowered by decreasing the dialysis time.

TABLE 5
The concentrations (μM) of amino acids of the
peritoneal fluid of the experimental group 3

| | Dialysis time (min) | | | |
|---|---|---|---|---|
| types | 0 | 3 | 5 | 10 |
| Asn | 30.674 | N/A | N/A | N/A |
| Arg | 83.86 | 70.12 | 37.31 | 19.99 |

According to Table 5 and compared with Table 4, the artificial kidney of the experimental group 3 has the effective membrane area of 2.1 m², which is larger than that of the experimental group 2, and Asparagine (Asn) is undetectable within 3 minutes of dialysis, and the limit of detection (LOD) of Asparagine was 5 μM. Hence, by using the artificial kidney with a larger effective membrane area, the removal of target amino acid could be speeded up.

Besides, the concentration of Arginine (Arg) is lower to 50% of the original concentration within 5 minutes in experimental group 3, and the concentration of Arginine is lower to 50% of the original concentration within 10 minutes in experimental group 2. Hence, by using the artificial kidney with larger effective membrane area, the removal of non-target amino acids will be speeded up as well. As the removal of non-target amino acids may incur side effect, the artificial kidney with the proper effective membrane area is suggested and further illustrated in Example 4.

### EXAMPLE 4

### Modified Peritoneal Dialysis

Example 4 was carried out with the method the same as Example 3 except that the artificial kidneys with different effective membrane areas were adopted. The concentrations of amino acids in the peritoneal fluid before and after dialysis were studied and the result was shown in Table 6.

TABLE 6
The concentrations (μM) of amino acids
of the peritoneal fluid of Example 4

| | | Dialysis time (min) | | | | |
|---|---|---|---|---|---|---|
| Dialyzer | Types | 0 | 3 | 5 | 10 | 20 |
| polyflux-140H | Asn (μM) | 36.9 | 31.6 | 23.0 | N/A | N/A |
| (1.4 m²) | Asn (%) | 100 | 85.8 | 62.3 | N/A | N/A |
| | Arg (μM) | 93.7 | 87.6 | 79.4 | 66.9 | 54.2 |
| | Arg (%) | 100 | 93.6 | 84.8 | 71.4 | 57.9 |
| BG-1.6U | Asn (μM) | 45.5 | 33.6 | N/A | N/A | N/A |
| (1.6 m²) | Asn (%) | 100 | 73.90 | N/A | N/A | N/A |
| | Arg (μM) | 95.3 | 82.2 | 71.3 | 62.7 | 47.4 |
| | Arg (%) | 100 | 86.3 | 74.8 | 65.8 | 49.7 |
| BG-1.8U | Asn (μM) | 36.7 | N/A | N/A | N/A | N/A |
| (1.8 m²) | Asn (%) | 100 | N/A | N/A | N/A | N/A |
| | Arg (μM) | 89.0 | 66.8 | 68.0 | 54.7 | 54.4 |
| | Arg (%) | 100 | 75.1 | 76.5 | 61.5 | 61.2 |

According to Table 6, by using the artificial kidney with a larger effective membrane area, the removal of target amino acid could be speeded up, while the concentration of non-target amino acid could be maintained at about 50% to reduce the risk of side effect.

Besides, the adaptation of the artificial kidney with the effective membrane area less than 1.4 m² requires longer time for dialysis. Hence, the artificial kidney with the effective membrane area within 1.4 m² to 1.8 m² was suggested.

### EXAMPLE 5

### Tumor Analysis

The present invention further comprises a tumor analysis before the extracorporeal dialysis, and the tumor analysis comprises the steps of (A1) to (A3) as follows:

(A1) obtaining a cancer cell from the patient: The cancer cell could be taken from the biopsy after surgery. Preferably, the cancer cell is a circulating tumor cell (CTC) collected from the blood sample of the patient. The collection method could be isolation by size of epithelial tumor cells (ISET) method or carried out by IsoFlux system of Fluxion Biosciences, Inc. or other commercially available technologies.

(A2) cultivating the cancer cell to obtain a cell culture: The cell culture could be a traditional two-dimensional (2D) culture. Preferably, the cell culture is a 3D cell culture. For example, the 3D cell culture could be a patient-derived three-dimensional organoid culture by using Matrigel to suspend the cell pellets. In the alternative, 3D scaffold system by the fabrication of 3D polycaprolactone (PCL) porous scaffolds is also available for culturing CTCs.

(A3) conducting an amino acid deprivation test to obtain a profile indicating the type of the target amino acid along with the corresponding concentration and period for cell death of the cell culture: After the 2D or 3D cell culture of the cancer cell from the patient is available, such cell culture is further divided for subculture in different levels and types of amino acid deprivation to acquire the data for the cell death of the cell culture. The data includes the types of the deprived amino acid, and the lethal low level of concentration and the period for the cell death of the cell culture. By analyzing such data, the vulnerability or sensitivity of the tumor could be utilized for the hemodialysis for removing target amino acid so as to kill the tumor specifically.

### EXAMPLE 6

### Blood Amino Acid Concentration Management

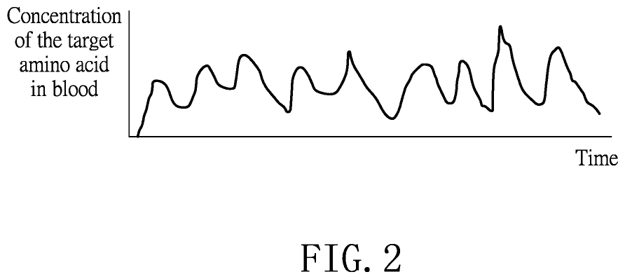

The concentration of the target amino acid in blood could be quantified by Ultra Performance Liquid Chromatography (UPLC). After the target amino acid is available from the tumor analysis, the blood amino acid concentration fluctuation as shown in FIG. 2 could be observed and a pattern analysis thereof could be executed before the extracorporeal dialysis. As the target amino acid in blood may increase sharply after meals, the timing to carry out extracorporeal dialysis to timely remove the target amino acid is calculated and decided according to the pattern of blood amino acid concentration fluctuation.

During the cancer treatment of the patient, continuous blood monitoring process for the concentration of the target amino acid is also required. Preferably, the data of the target amino acid concentration is received in a real time manner during the extracorporeal dialysis and periodically during the intervals of extracorporeal dialysis. In the alternative, the frequency to get the data of the target amino acid concentration could be reduced and said data could be acquired within 2 hours after a meal and whenever the extracorporeal dialysis is completed only.

Further, the blood monitoring process could be provided to track compensatory effect due to the deprivation of the target amino acid.

In order to timely remove the target amino acid at an accurate clearance rate, nutrition management for the patients is provided during the cancer treatment, so that the concentration of the target amino acid after meals could be well-controlled to stay within an acceptable fluctuation range. Further, the nutrition management will not remove the food releasing the target amino acid from daily diet of patients completely.

Preferably, the data to draw the blood amino acid concentration fluctuation as shown in FIG. 2 is acquired under the condition that the patient follows the instruction the same as the nutrition management, so that the pattern analysis aforementioned could be more precise and reliable. To sum up, the present invention modifies dialysis to achieve in vitro amino acid deprivation and further incorporates personalized diagnosis, the present invention not only provides a novel precision medicine with better anticancer efficacy and less side effects, but also requires less time and cost for development. Even though numerous characteristics and advantages of the present invention have been set forth in the foregoing description, together with details of the steps and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for cancer treatment in a patient comprising extracorporeal dialysis of blood, plasma or peritoneal fluid of the patient with a dialysis system for removing a target amino acid, and the dialysis system comprising: a dialysis machine, a dialyzer having a dialysis membrane, and a dialysate; wherein the dialyzer is connected to the dialysis machine and the dialysate flows within the dialysis machine and the dialyzer; and an enzyme for degrading the target amino acid is provided to the dialysis membrane and/or the dialysate, and the target amino acid includes asparagine, glutamine, arginine, serine, methionine or any combination thereof.

2. The method as claimed in claim 1, wherein the dialysate is in an amount of 1 time to 9 times of the amount of the blood, the plasma or the peritoneal fluid of the patient.

3. The method as claimed in claim 1, wherein the flow rate or circulation speed of the dialysate is 200 ml/min to 600 ml/min.

4. The method as claimed in claim 1, wherein the concentration of the enzyme for degrading the target amino acid in the dialysate is more than 0.1 K.U./ml.

5. The method as claimed in claim 1, wherein the dialyzer has an effective membrane area more than 0.5 m².

6. The method as claimed in claim 1, wherein the extracorporeal dialysis requires 3 minutes to 1 hour for each time.

7. The method as claimed in claim 1, wherein the extracorporeal dialysis is carried out periodically or irregularly and lasts for 3 days to 2 weeks.

8. The method as claimed in claim 1, wherein the cancer is selected from a group consisting of acute lymphoblastic leukemia (ALL), acute myeloid leukemia (AML), small cell lung cancer, lung mesothelioma, chest wall tumor, abdomen-pelvis tumor, small intestinal tumor, thymoma, mediastinal tumor, prostate cancer, cervical cancer, endometrial cancer, ovarian cancer, uterine sarcoma blood cancer, lymphoma, pancreatic cancer, sarcoma, brain cancer, low-grade astrocytoma, high-grade astrocytoma, pituitary adenoma, meningioma, CNS lymphoma, oligodendroglioma, brain stem tumor, ependymoma, breast cancer, colorectal cancer, liver cancer, liver adenocarcinoma, gallbladder cancer, biliary tract cancer, head and neck cancer, nasopharyngeal cancer, esophageal cancer, craniopharyngioma, laryngeal cancer, oropharyngeal cancer, salivary gland tumor, hypopharyngeal cancer, thyroid cancer, oral cavity tumor, kidney cancer, bladder cancer, anal cancer and skin cancer.

9. A method for personalized cancer treatment by removing a target amino acid in a patient, which comprises:
a tumor analysis comprising the steps of:
(A1) obtaining a cancer cell from the patient;
(A2) cultivating the cancer cell to obtain a cell culture; and
(A3) conducting an amino acid deprivation test to obtain a profile indicating the type of the target amino acid along with the corresponding concentration and period for cell death of the cell culture, wherein the target amino acid includes asparagine, glutamine, arginine, serine, methionine or any combination thereof;
a pattern analysis of blood concentration fluctuation of the target amino acid;
extracorporeal dialysis of blood, plasma or peritoneal fluid of the patient with a dialysis system for removing the target amino acid, and the dialysis system comprising: a dialysis machine, a dialyzer having a dialysis membrane, and a dialysate; wherein the dialyzer is connected to the dialysis machine and the dialysate flows within the dialysis machine and the dialyzer; and an enzyme for degrading the target amino acid is provided to the dialysis membrane and/or the dialysate, and the dialyzer is a high flux dialyzer;
a blood monitoring process for the concentration of the target amino acid;
nutrition management during the cancer treatment of the patient;
wherein the pattern analysis is carried out before the extracorporeal dialysis, and the extracorporeal dialysis is carried out stage by stage; and
a tumor tracking process during the personalized cancer treatment of the patient.

10. A hemodialysis system comprising: a dialysis machine, a dialyzer having a dialysis membrane, and a dialysate; wherein the dialyzer is connected to the dialysis machine and the dialysate flows within the dialysis machine and the dialyzer; and an enzyme for degrading a target amino acid is provided to the dialysis membrane and/or the dialysate, and the target amino acid includes asparagine, glutamine, arginine, serine, methionine, or any combination thereof.
